(19) United States
(12) Patent Application Publication
(10) Pub. No.: US 2005/0100568 A1
(43) Pub. Date: May 12, 2005

(54) Cosmetic compositions and methods for reducing the appearance of pores

(75) Inventors: Marc Nicolaas De Mul, Hoboken, NJ (US); Helen Elizabeth Knaggs, Weehawken, NJ (US); Robert Edward Marriott, Edgewater, NJ (US); Joseph Raymond Faryniarz, Middlebury, CT (US)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: Unilever Home & Personal Care USA, Division Of Conopco, Inc.

(21) Appl. No.: 10/706,756

(22) Filed: Nov. 12, 2003

(51) Int. Cl. A61K 7/021; A61K 7/06; A61K 7/11

(52) U.S. Cl. 424/401; 424/70.12; 424/63

(57) ABSTRACT

A cosmetic composition and method for reducing the appearance of pores. The composition is based on a silicone elastomer or a water soluble or dispersible polymer and light scattering particles, and having a specified unique rheological profile. The inventive compositions and methods deliver an improvement in appearance of pores of at least about 2, preferably at least about 4, as measured by the Pore Ruler. Pores appear less significant and smaller in size, while natural skin tone is maintained.

### FIELD OF THE INVENTION

The present invention relates to a cosmetic composition and method for cosmetically reducing the appearance of skin pores, while maintaining natural skin tone and minimizing the sensation of stickiness, tackiness, and skin tightness.

### BACKGROUND OF THE INVENTION

The ultimate goal of a cosmetic composition for reducing the appearance of pores, as well as that of any cosmetic product or method, is a satisfied consumer. Many cosmetic products advertise facial pore benefits. However, it is often difficult to discern the extent to which a benefit is delivered and, even if delivered, undesirable sensory effects accompany the benefits. Undesirable sensory effects include stickiness, tackiness, skin tightness, and skin opacifying effects.

Make-up cosmetics for reducing the appearance of fine lines and wrinkles, employing silicone elastomers in combination with spherical particles are disclosed in European Patent Application EP 1 136 064. U.S. Pat. No. 6,027,738 relates to anhydrous makeup comprising silicone gel, containing organosilicone elastomer dispersed in silicone-compatible vehicle, and a silicone oil base. EP 0 826 364 discloses an oily cosmetic powder containing silicone elastomers. A need remains for commercially acceptable compositions for reducing the appearance of pores, while maintaining natural skin tone, but that do not impart stickiness, tackiness, skin tightness, or skin opacifying or whitening effects.

### SUMMARY OF THE INVENTION

Cosmetic compositions that yield visible pore size reduction while maintaining natural skin appearance have been developed. The inventive compositions are characterized by low Opacity, low Gloss, and a shear-thinning rheology with a relatively large normal force at high shear. The compositions according to the present invention include:
(a) about 0.01 to about 10% by weight of the composition of a water based polymer or about 0.01 to about 30% cross-linked polysiloxane elastomer;
(b) about 0.1% to about 25% of light scattering particles, i.e. dispersed solid particles; and
(c) a cosmetically acceptable vehicle;
having:
   a viscosity at room temperature of about 10 Pa.s to about 100 Pa.s at a shear rate of 1 1/s; and about 0.01 Pa.s to about 0.3 Pa.s at 10,000 1/s;
   an Opacity of about 3% to about 6%;
   a 60 degrees Gloss of up to about 10 g.u.;
   a normal force of up to about 0.1 N at a shear rate of 10,000 1/s; and

resulting in an improvement of appearance of facial pores of at least about 2 as measured by the Pore Ruler. The improvement can be as high as about 4 as measured by the Pore Ruler.

### DETAILED DESCRIPTION OF THE INVENTION

The present invention meets the needs left unmet by prior art, by providing compositions and methods for cosmetically reducing the appearance of facial pores. The present invention is directed to compositions including a silicone elastomer or water based polymer and light scattering particles dispersed therein. The compositions form a layer with structures within it upon application to skin and yield an improvement in the appearance of pores of at least about 2 as measured by the Pore Ruler.

The inventive compositions are characterized by shear-thinning rheology with a relatively large normal force at high shear. To avoid the sensation of tackiness, the inventive compositions have a viscosity at room temperature (about 25 C) of about 10 Pa.s to about 100 Pa.s at a shear rate of 1 1/s; and about 0.01 Pa.s to about 0.3 Pa.s at 10,000 1/s; as well as a normal force (Fₙ) of up to about 0.1 N at a shear rate of 10,000 1/s.

To enhance natural skin tone, the inventive compositions have an Opacity of about 3% to about 6%. The Gloss of the compositions, at 60 degrees, is up to about 10 g.u. (gloss units).

Except in the operating and comparative examples, or where otherwise explicitly indicated, all numbers in this description indicating amounts of material or conditions of reaction, physical properties of materials and/or use are to be understood as modified by the word “about.” All amounts are by weight of the final composition, unless otherwise specified.

As used herein, the term “comprising” means including, made up of, composed of, consisting and/or consisting essentially of.

The term “skin” as used herein includes the skin on or in the face, mouth, neck, chest, back, arms, hands, legs, and scalp.

As used herein, skin pores are defined as openings or troughs on the skin surface. More particularly, a pore is an opening for a sebaceous oil gland. Pores are microscopic openings in skin that provide a way for oil or sebum to lubricate and protect the skin surface. Glands enlarge during puberty and there is a concomitant increase in the amount of oil produced. Consumers report that their pores get bigger to handle the increased output, although the true mechanisms controlling pores remain unknown at present. The overall appearance of pores depends on the depth and diameter of the troughs as well as on the surrounding skin color, texture and periodicity of the pores.

### Polymer

Film forming polymers are used in the compositions according to the present invention, to form a layer of film over pores when deposited on the skin.

### Elastomer Polymers

According to a first embodiment of the present invention, cross-linked silicone elastomers are employed. A cross-linked silicone elastomer forms a film, such as over pores, the appearance of which it evens out. The cross-linked silicone elastomer also imparts silkiness to the skin.

Silicone elastomers are blends of highly cross-linked siloxane polymers (cross-linked polysiloxane elastomers) and silicone oils. Supplier sources include GE Silicones (Waterford, N.Y.) and Dow Corning (Midland, Mich.). Elastomers are preferably included in an amount of about 0.01% to about 30%, preferably about 1% to about 10%.

Most preferably, to help disperse the elastomer uniformly in the formulations, the elastomer is included in combination with additional silicone oils (cyclomethicones and dimethicones). In that case, the silicone oil is included in an amount of about 0% to about 80%. Additionally, the oil imparts good skin feel and emolliency.

TABLE 1
Silicone Elastomer Materials

| Brand Name | Manufacturer | INCI name |
|---|---|---|
| KSG-15 | Shin-Etsu | Dimethicone/Vinyl dimethicone crosspolymer |
| KSG-16 | Shin-Etsu | Dimethicone/Vinyl dimethicone crosspolymer |
| KSG-18 | Shin-Etsu | Dimethicone/Vinyl dimethicone crosspolymer |
| SFE818 | GE Silicones | Cetearyl Dimethicone/Vinyl Dimethicone Crosspolymer |
| SFE839 | GE Silicones | Dimethicone/Vinyl dimethicone crosspolymer |
| Velvesil 125 | GE Silicones | C30-45 Alkyl Cetearyl Dimethicone Crosspolymer |
| Gransil GCM | Grant Industries | Polysilicone-11 |
| Gransil DMG-6 | Grant Industries | Polysilicone-11 |
| Gransil PM Gel | Grant Industries | Polysilicone-11 |
| 9011 | Dow Corning | PEG-12 Dimethicone Crosspolymer |
| 9040 | Dow Corning | Dimethicone Crosspolymer |
| 9041 | Dow Corning | Dimethicone Crosspolymer |
| 9045 | Dow Corning | Dimethicone Crosspolymer |
| 9506 | Dow Corning | Dimethicone/Vinyl Dimethicone Crosspolymer |
| 9509 | Dow Corning | Dimethicone/Vinyl Dimethicone Crosspolymer; C12-14 |
| | | Pareth-12 |
| 9546 | Dow Corning | Dimethicone Crosspolymer; Dimethicone/Vinyl Dimethicone |
| | | Crosspolymer; Dimethiconol |
| Trefil E-506 S | Dow Corning | Dimethicone/Vinyl Dimethicone Crosspolymer |

Water Based Polymer

Film forming polymers, according a second embodiment of the present invention, are selected in such a way as to reduce the appearance of facial pores while minimizing the perception of stickiness, tackiness, and tightness that may be experienced with certain compositions.

To avoid application of too thick a film on the facial pores, the inventive compositions include no more than about 10% film forming polymer.

TABLE 2
Water-based Polymer Film Former Materials

| Tradename | INCI name | Supplier |
|---|---|---|
| Luviflex Soft | Acrylates Copolymer | BASF |
| Luviform FA139 | PVM/MA copolymer | BASF |
| Eastman AQ38S | Diglycol/CHDM/Isophthalates/SIP Copolymer | Eastman |
| | | Chemical |
| Eastman AQ48 Ultra | Diglycol/CHDM/Isophthalates/SIP Copolymer | Eastman |
| | | Chemical |
| Eastman AQ55S | Diglycol/CHDM/Isophthalates/SIP Copolymer | Eastman |
| | | Chemical |
| Aquaflex FX-64 | Isobutylene/ethylmaleimide/hydroxyethylmaleimide | ISP |
| | copolymer | |
| Gafquat 755N | Polyquaternium-11 | ISP |
| Gantrez S-97 BF | 2-butenedioic acid, polymer with methoxyethene | ISP |
| PVP K-90 | PVP | ISP |
| PVP/VA W-735 | PVP/VA copolymer | ISP |
| Styleze 2000 | VP/acrylates/lauryl methacrylate copolymer | ISP |
| Styleze CC-10 | VP/DMAPA acrylates copolymer | ISP |
| Styleze W-20 | Polyquaternium-55 | ISP |
| Amaze XT | Corn Starch Modified | National |
| | | Starch |
| Flexan 130 | Sodium polystyrene sulfonate | National |
| | | Starch |
| Dermacryl LT | Acrylates/Octylacrylamide Copolymer | National |
| | | Starch |
| Avalure AC-120 | Acrylates copolymer | Noveon |
| Avalure UR-425 | Polyurethane-2 dispersion | Noveon |
| Avalure AC-118 | Acrylates copolymer | Noveon |
| Avalure AC-122 | Acrylates copolymer | Noveon |
| Avalure UR-445 | Polyurethane-2 dispersion | Noveon |
| Fixate G-100 | AMP-Acrylates/Allyl Methacrylate Copolymer | Noveon |
| Silsoft Surface Film Former | Dimethicone PEG-8 Polyacrylate | OSi Specialties |
| Jaguar C162 | Hydroxypropyl Guar Hydroxypropyltrimonium Chloride | Rhodia |
| Jaguar HP-105 | Hydroxypropyl Guar | Rhodia |

Light Scattering Particles

Light scattering particles according to the present invention may be spherical or non-spherical particles of less than about 30 micro-meters in size, preferably about 200 nm to about 20 micro-meters, for optimum scattering. The light scattering particles may be pigments, mattifiers, or fillers. Metal oxides (e.g. titanium dioxide), mineral oxides (e.g. silica, mica (e.g. titanium coated mica), talc), nylon, and polymethyl methacrylate cross-polymer (e.g. GANZPEARL brand, PMMA) are examples of suitable light scattering particles. The particles may be surface coated or surface treated.

The amount of light scattering particles in the cosmetic composition is preferably in the range of about 0.1% to about 25%, more preferably about 1% to about 10%, most preferably about 1% to 5%, depending on the light scattering properties of the particular material. The light scattering particles and the polymers are present in the composition in a weight ratio of about 1:10000 to about 10000:1 of light scattering particle: polymer, preferably about 10:1 to about 1:100.

The light scattering particles may be water dispersible or oil dispersible. For example, water-dispersible titanium dioxide, in accordance with the invention, is micronized titanium dioxide, the particles of which are uncoated or which are coated with a material to impart a hydrophilic surface property to the particles. Examples of such materials include aluminum oxide and aluminum silicate. Oil-dispersible titanium dioxide, in accordance with the invention, is micronized titanium dioxide, the particles of which exhibit a hydrophobic surface property.

TABLE 3
Light Scattering Particles

| Name | Manufacturer | INCI name |
|---|---|---|
| Cab-o-sil L-90 | Cabot | Silica |
| Cab-o-sil M-5 | Cabot | Silica |
| Cab-o-sil TS-720 | Cabot | Silica |
| Aerosil R974 | Degussa | Silica dimethyl silylate |
| FK500LS | Degussa | Silica |
| Sipernat ® 500 LS | Degussa | Silica |
| TOSP2000B | GE Silicones | Polymethylsilsesquioxane |
| Tospearl ® 120A | GE Silicones | Polymethylsilsesquioxane |
| Tospearl ® 130A | GE Silicones | Polymethylsilsesquioxane |
| Tospearl ® 145A | GE Silicones | Polymethylsilsesquioxane |
| BPA-5I3 | Kobo | Polymethyl Methacrylate |
| BPA-5I5 | Kobo | Polymethyl Methacrylate |
| BTD-401 | Kobo | Titanium dioxide |
| DSPCS/3H-12 | Kobo | Silica/EMA/PMMA-isopropyl Ti Stearate |
| ES-830 | Kobo | Polymethyl Methacrylate |
| Mica S-I2 | Kobo | Mica |
| MSS-500/3H | Kobo | Silica |
| TP-145A | Kobo | Silicone resin |
| Dry-Flo AF | National | Modified corn starch |
| | Starch | |
| Tapioca Pure | National | Tapioca starch |
| | Starch | |
| Cosmo-55 | Presperse | Silica |
| Ganzpearl GM 0830 | Presperse | PMMA |
| Ganzpearl GMP | Presperse | PMMA |
| 0820 | | |
| Ganzpearl GMX | Presperse | PMMA |
| 0610 | | |
| Ganzpearl GMX | Presperse | PMMA |
| 0810 | | |
| Ganzpearl GPA-550 | Presperse | Nylon-12 |
| Ganzpearl GS 0605 | Presperse | Styrene |
| Ganzpearl PS-8F | Presperse | Styrene/DVB Copolymer |
| MCP-45 | Presperse | Mica; poly (alkyl acrylate) |
| Micro-Ace P-2-030 | Presperse | Talc; methicone |
| Micro-poly 220L | Presperse | Polyethylene |
| Micro-poly 250S | Presperse | Polyethylene |
| Rose Talc | Presperse | Talc |
| SM-1000 | Presperse | Mica; silicic anhydride |
| SM-2000 | Presperse | Mica; silicic anhydride |
| SM-4000 | Presperse | Mica; silicic anhydride |
| SP-29 UVS | Presperse | Bismuth oxychloride; sericite; silicic anhydride |
| Talc Micro-Ace P-2 | Presperse | Talc |
| Velvet Veil 310 | Presperse | Mica; silicic anhydride |
| SunPMMA-S | Sunjin | PMMA |
| Sunsil-130H | Sunjin | Silica |
| Micro TiO2 MT- | Tri-K | Titanium dioxide |
| 100SA | | |
| Neosil CT11 | | Silica |

Optional Ingredients

Various types of active ingredients may be present in cosmetic compositions of the present invention. Actives are defined as skin or hair benefit agents other than emollients and other than ingredients that merely improve the physical characteristics of the composition. Although not limited to this category, general examples include sunscreens, skin lightening agents, tanning agents, niacinamide, vitamins, and antioxidants.

Sunscreens include those materials commonly employed to block ultraviolet light. Illustrative compounds are the derivatives of PABA, cinnamate and salicylate. For example, octyl methoxycinnamate and 2-hydroxy-4-methoxy benzophenone (also known as oxybenzone) can be used. Octyl methoxycinnamate and 2-hydroxy-4-methoxy benzophenone are commercially available under the trademarks, Parsol MCX and Benzophenone-3,respectively. The exact amount of sunscreen employed can vary depending upon the degree of protection desired from the sun's UV radiation.

Another preferred optional ingredient is selected from essential fatty acids (EFAs), i.e., those fatty acids which are essential for the plasma membrane formation of all cells. In keratinocytes EFA deficiency makes cells hyper-proliferative. Supplementation of EFA corrects this. EFAs also enhance lipid biosynthesis of epidermis and provide lipids for the barrier formation of the epidermis. The essential fatty acids are preferably chosen from linoleic acid, gamma-linolenic acid, homo-gamma-linolenic acid, columbinic acid, eicosa-(n-6,9,13)-trienoic acid, arachidonic acid, gamma-linolenic acid, timnodonic acid, hexaenoic acid and mixtures thereof.

Other optional ingredients may include herbal extracts, anti-oxidants, coloring agents and perfumes. Amounts of these materials may range anywhere from 0.001% up to 20% by weight of the composition.

An oil or oily material may be present, together with an emulsifier, to provide either a water-in-oil or an oil-in-water emulsion, depending largely on the average hydrophilic-lipophilic balance (HLB) of the emulsifier employed.

### Cosmetically Acceptable Vehicle

The composition according to the invention also comprises a cosmetically acceptable vehicle to act as a dilutant, dispersant or carrier for the active components in the composition, so as to facilitate their distribution when the composition is applied to the skin or hair.

Vehicles other than or in addition to water can include liquid or solid emollients, solvents, humectants, thickeners and powders. An especially preferred nonaqueous carrier is a polydimethyl siloxane and/or a polydimethyl phenyl siloxane. Other preferred nonaqueous carriers are cyclic polydimethyl siloxanes, such as octamethyl cyclotetrasiloxane or decamethyl cyclopentasiloxane. Silicones of this invention may be those with viscosities ranging anywhere from about 10 to 10,000,000 centistokes at 25° C. Especially desirable are mixtures of low and high viscosity silicones. These silicones are available from the General Electric Company under trademarks Vicasil, SE and SF and from the Dow Corning Company under the 200 and 550 Series. Amounts of silicone which can be utilized in the compositions of this invention range anywhere from about 5 to about 95% by weight of the composition.

A humectant may be included in the inventive compositions to deliver a moisturizing benefit to the skin. Suitable humectants are polyhydric alcohols and include, but are not limited to glycerol (a.k.a. glycerin), humectants other than glycerin which can be added herein include sorbitol, propylene glycol, butylene glycol, hexylene glycol, ethoxylated glucose and hexanetriol. Humectants, such as glycerol and sorbitol, are known as excellent moisturizers for skin, scalp and hair. See for instance WO9111171, WO9219216, WO219275 and U.S. Pat. No. 5,858,340.

The moisturizing capabilities of these ingredients are dependent. If the inventive compositions include a humectant, it is included at a concentration of at least about 1%. Generally, the concentration of humectant is in the range from about 1% to about 90%, preferably from about 1% to about 15%, to optimize the moisturizer benefits to the skin and plasticizing effects on the composition. The most preferred humectants are glycerol and sorbitol due to their low cost and high efficacy.

In water based compositions according to the present invention, a thickener is optional, but preferred. Thickeners are used in the inventive composition in an amount of up to about 2% by weight of the composition. Examples of suitable thickeners are listed in the table below.

TABLE 4
Thickeners and Rheological Additives

| Tradename | INCI name | Supplier |
|---|---|---|
| Carbopol ETD 2020 | Carbomer | Noveon |
| Keltrol CG | Xanthan gum | CP Kelco |
| Aristoflex AVC | Ammonium | Clariant |
| | Acryloyldimethyltaurate/ | |
| | VP Copolymer | |
| Structure ZEA | Hydroxypropyl Starch Phosphate | National Starch |
| Celquat SC-230M | Polyquaternium-10 | National Starch |

In water based compositions according to the present invention, a neutralizing agent is optional, but is preferably included to neutralize fatty acids, thereby building viscosity and stabilizing emulsion structure. Suitable neutralizing agents include but are not limited to triethanolamine, potassium hydroxide, sodium hydroxide, ammonium hydroxide, and amino methyl propanol ethanolamine. The neutralizing agent is preferably included in an amount of about 0% to about 5%, most preferably in an amount of about 0.05% to about 1%.

Use of the Composition

The composition according to the invention is intended primarily as a product for topical application to human skin, especially as an agent for reducing the appearance of facial skin pores.

In use, a small quantity of the composition, for example from 1 to 5 ml (milli-liters), is applied to exposed areas of the skin or hair from a suitable container or applicator. If necessary, it is then spread over and/or rubbed into the skin or hair using the hand or fingers or a suitable device.

In accordance with the present invention, after a single application, the compositions of the present invention result in reduction of appearance of facial skin pores of at least about 2, as measured by the Pore Ruler.

Pore Ruler measurements were used as the experimental technique to objectively measure pore size appearance before and after application of the compositions according to the present invention, and according to the inventive methods. The Pore Ruler technique is described in more detail hereinbelow, with reference to its development and utility as an objective measure of appearance of pore size.

### Pore Ruler

The Pore Ruler is a test device for objectively determining facial skin pore condition and/or measuring progress or efficacy of the cosmetic composition over a prolonged period of its application.

The pore ruler device is based on the establishment of a clinical grading scale relevant to consumer perception. Accordingly, the pore ruler device is a consumer perceivable, clinically usable tool for the assessment of facial pores. It may be applied for consumer self-evaluation or for evaluation by a beautician or sales associate.

The Pore Ruler is based on an empirically derived scale for objectively assessing attributes of pores on an area of human skin. The pore ruler device is based on a 9 point scale. Essentially, the following steps were taken to develop the Pore Ruler:
1. Photography
2. Sorting
3. Analysis of Sorted Data
4. Selection of Images
5. Validation of Draft Pore Ruler by Consumers
   1. Photography

100 Japanese female subjects were photographed using a digital camera under the same lighting conditions and using the same camera settings. The women were asked to remove their facial makeup before being photographed. The photos were obtained in 3 views, the left, front and right views of each subject. The digital images were then burnt on a CD-R and consistently printed on A4 size photo paper on the Codonics® color printer. The settings on the printer were optimized to closely match the color of the images when viewed on a BARCO® calibrated color monitor.

It is preferable to grey out the eyes to eliminate possible identification of the subjects. Even more preferable is to transform the images of the pores on a single face, to facilitate comparison of pore attributes without distraction caused by variation of unrelated facial features.

2. Sorting

The 100 printed photographs were placed into 9 bins by evaluators both in the United States as well as in Japan in increasing order of perceivable pore size or “pores standing out”. There were 6 evaluators in the U.S. and 3 in Japan who participated in this exercise and it took about 1.5 to 3 hours per evaluator to complete the exercise. The evaluators adopted one of many ways to achieve the sorting. Whereas a few evaluators started off by splitting the stack into 3 bins (i.e.) low, medium and high, before further classifying each of the bins until they arrived at the final result of 9 bins. Other evaluators began by identifying the extremes in condition of perceivable pore size, and then worked their way through the middle. Irrespective of the method adopted, the evaluators were always instructed to end up with 9 pore bins that were representative of differences in perceivable pore size. In addition, the evaluators were also asked to identify one single photograph per bin that was most representative of the images in that bin.

3. Analysis of Sorted Data

About 90% of the evaluators were in agreement among themselves within one “pore bin”. The next step in the development of the ruler is the selection of images.

4. Selection of Images

The selection of images for the pore ruler consisted of the following steps:
1. Select “representative” images out of each bin (draft pore-ruler)
2. Attain high agreement (low standard deviations) as to the selected images across the different evaluators
3. Select those images that provide equal steps between neighboring bins
4. Iterate between Japanese and U.S. graders until agreement is reached.

In co-pending U.S. patent application Ser. No. 10/606,390, hereby incorporated by reference herein, FIG. 1 represents a Pore Ruler having 9 images. The Pore Ruler images capture a range of conditions in facial pores that are representative facial pores of Japanese consumers.

5. Validation of Draft Pore Ruler

The next step in the development of the ruler was the validation of the draft ruler by consumers. This involved two steps. The first step was the validation of the ruler by clinicians. The results from this validation showed that the rank ordering of the pore ruler images by the clinicians is the same as that of the graders both in Japan and the U.S.

The next step in the validation process involved presenting the images to naive Japanese consumers and having them rank order the images 55 Japanese consumers (age: 20-55, female) were asked to rank the photographs in increasing order of perceivable pore size. The consumers ranked the images in the same order as the clinicians. There was significant difference for every pair of photos at 95%.

A one point improvement on the scale was considered to be both noticeable and relevant to the consumer as well as the expert grader.

The results above show that the Pore Ruler is a consumer perceivable ruler, and can be used in studies for visual assessment of the pores in an objective manner.

### Product Form and Packaging

The composition can be packaged in a suitable container to suit its viscosity and intended use by the consumer. For example, a composition can simply be stored in a non-deformable bottle or squeeze container, such as a lidded jar or a tube.

The invention accordingly also provides a closed container containing a cosmetically acceptable composition as herein defined.

A cosmetic product system, including a cosmetic composition packaged together with a simple diagnostic pore ruler device, is another embodiment of the present invention.

The following specific examples further illustrate the invention, but the invention is not limited thereto.

### EXAMPLE 1

The formulations detailed in the Table below were prepared using the following procedures:

All preparation was performed at room temperature using overhead mixers (1000 rpm).

In the Table below, the compositions are listed as the percentage active, not the percentage samples as received. For example, 70 weight percent of Dow Corning 9040 Silicone Elastomer Blend was used in Example 1. This material consists of 12 to 13 weight percent silicone elastomer polymer and 87 to 88 weight percent cyclopentasiloxane, which is equivalent to Dow Corning 245 Fluid. Therefore, the content of silicone elastomer polymer in the Example 1 formulation is 8.75 weight percent and the content of cyclopentasiloxane is 61.25 weight percent.

Compositions 1-3

The formulations 1, 2, and 3 were prepared by combining the Dow Corning 9040 Silicone Elastomer Blend and the Dow Corning 200 Fluid, followed by stirring using an overhead mechanical stirrer until homogeneous. The particulate additives are slowly mixed in, followed by agitation for 20 minutes or more to obtain a smooth, homogeneous dispersion.

5 Composition 4

Carbopol ETD2020 was dispersed in water using an overhead mixer. The particulate additive, in this case Neosil CT11, was slowly added and the mixture was homogenized for 20 minutes at high shear. The pH of the dispersion was then adjusted to a pH above 5 using a NaOH solution. Finally, the Silsoft Surface Film Former was added and the mixture was homogenized using an overhead mixer until a smooth, homogeneous dispersion was obtained.

Composition 5

Rhodopol 23 powder was dispersed in 1,3-butylene glycol, followed by slowly adding water while stirring. A homogeneous dispersion was obtained with a Rhodopol 23 concentration of 2 weight percent, a 1,3-butylene glycol concentration of 5 weight percent, and the balance water. This dispersion was added to a mixing vessel in an amount sufficient to make the Rhodopol 23 concentration in the fully formulated mixture equal to 0.50 weight percent. Water and glycerol were added and the mixture was stirred using an overhead mechanical stirrer at 800 rpm until the Rhodopol 23 was fully dissolved. At that time, Neosil CT11 was added slowly to avoid coagulation and the dispersion was homogenized using the overhead stirrer. Finally, Flexan II powder was added and the formulation stirred until homogeneous.

Composition 6

Styleze CC-10, glycerol, and water were added to a mixing vessel and slowly agitated with an overhead mechanical stirrer until homogeneous. Celquat 230M powder was added and dissolved. Neosil CT11 was added and the dispersion was agitated with the overhead stirrer until smooth and homogeneous.

Composition 7

Carbopol ETD2020 was dispersed in water using an overhead mechanical stirrer, followed by addition of Micro Titanium Dioxide MT-100SA and stirring until well dispersed. The viscosity of the dispersion was increased by neutralizing the Carbopol ETD2020 by addition of sodium hydroxide solution until the pH equaled about 6. Avalure AC120 was added and the resulting dispersion mixed until homogeneous.

Compositions 8, 9, 11

Formulation procedures similar to Compositions 1-3.

Composition 10

Formulation procedure similar to Composition 6.

Compositions 1-5 in the Table 5 are within the scope of the invention, while compositions 6 -11 are outside the scope of the invention, as shown by the physical properties in the Table 6 below.

Optical and rheological properties of the compositions were measures according to the following procedures.

Opacity. A Hunterlab LabScan XE automated spectrophotometer was used to measure the opacity of product coatings. The coatings were made on Leneta Form 2A opacity test charts held in place on a vacuum plate, and an 8-path wet film applicator was used to coat a film with a wet thickness of 2 mils, i.e. 50.8 μm (all equipment supplied by Paul N. Gardner Co., Pompano Beach, Fla.). This wet film thickness was chosen to approximate the film thickness in the clinical tests, in which 75 μL was applied on about 2 in.², i.e. 1290 mm², which corresponds to a wet film thickness of 58 μm. The coatings were air dried before the opacity measurement.

The opacity values were reported as percent opacity, defined as the Y value of the coating on the black area of the test chart divided by the Y value on the white area times 100%. The Y value is the CIE Tristimulus Y coordinate measured by the Hunterlab instrument. If a coating were fully transparent, the opacity would be 0%; if fully opaque, it would be 100%.

Gloss measurements. The specular gloss of product coatings was measured using a commercial glossmeter, the Rhopoint 20°/60°/85° Novo-Gloss Statistical Glossmeter (Rhopoint Instrumentation, Ltd., United Kingdom). Specular gloss is a measure of the shininess of a product film. It is defined in international standards as the ratio of the luminous flux reflected from an object in a specular direction, for a specified source and receptor angle, to the luminous flux reflected from glass with a refractive index of 1.567 in a specular direction.

To define the gloss scale, by convention a value of 100 gloss units is assigned to polished black glass with n_{D =}1.567 for reflection angles of 20°, 60°, and 85°.The glossmeter used is supplied with two gloss calibration standards, one with zero gloss and one with a high gloss of 93.2 g.u. at 60°. It was found that a reflection angle of 60° yielded the largest separation in gloss values between samples, and all subsequent measurements were made at that angle.

Gloss measurements were made on product films coated on opacity test charts using the same procedure as in the opacity measurements.

Rheology measurements. The rheology of the compositions was measured using a controlled strain rheometer (ARES, Rheometric Scientific, Piscataway, N.J.). The test geometry used here was a 25 mm diameter parallel plate at a sample gap thickness of 100 μm. The shear rates available in this geometry depend on the rotation rate and the sample gap thickness. With a 100 μm gap, a maximum shear rate of 10,000 to 100,000 1/s is achievable. All measurements were done at 25° C.

Small volumes of the samples were deposited between the plates and squeezed to the 100 μm thickness. A force gap test mode was used which controlled the maximum force exerted on the sample as the parallel plates were brought together. Excess sample material at the plate edge was removed to improve reproducibility.

The samples were sheared starting at a shear rate of 1 1/s. The shear rate was increased stepwise until it reached 10,000 1/s and the viscosity and normal force of the sample were recorded. After the measurement was complete, the normal force was corrected for baseline. The values of the viscosity at 1 1/s and 10,000 1/s and of the normal force at 10,000 1/s were taken as characteristic of the sample.

TABLE 5

| | (Amounts in | | | | | | | | | | | | |
|---|---|---|---|---|---|---|---|---|---|---|---|---|---|
| | weight percent.) | | Inventive | | | | | | | | | | |
| Formulation | | Ingredient | Compositions | | | | | Comparative | | | | | |
| Ingredients | % active | manufacturer | 1 | 2 | 3 | 4 | 5 | 6 | 7 | 8 | 9 | 10 | 11 |

Polymers

| | | | | | | | | | | | | |
|---|---|---|---|---|---|---|---|---|---|---|---|---|
| 9040 Silicone | 12.5 | Dow Corning | 8.75 | | 6.25 | | | | | 10.00 | 6.25 | |
| Elastomer | | | | | | | | | | | | |
| Blend | | | | | | | | | | | | |
| Velvesil 125 | 12.5 | General | | 10.00 | | | | | | | | |
| | | Electric | | | | | | | | | | |
| Silsoft Surface | 25 | OSi Specialties | | | | 5.00 | | | | | | |
| Film Former | | | | | | | | | | | | |
| Flexan II | 100 | National Starch | | | | | 2.92 | | | | | |
| Styleze CC-10 | 10 | ISP | | | | | | 3.00 | | | | 3.00 |
| Avalure | 29 | Noveon | | | | | | | 2.00 | | | |
| AC-120 | | | | | | | | | | | | |

Thickeners

| | | | | | | | |
|---|---|---|---|---|---|---|---|
| Carbopol | 100 | Noveon | 0.25 | | | 0.25 | |
| ETD 2020 | | | | | | | |
| Rhodopol 23 | 100 | Rhodia | | 1.00 | | | |
| Celquat | 100 | National Starch | | | 1.00 | | 0.25 |
| SC230M | | | | | | | |

Vehicle

| | | | | | | | | | | | | | |
|---|---|---|---|---|---|---|---|---|---|---|---|---|---|
| 1,3-Butylene | 100 | | | | | | 2.50 | | | | | | |
| glycol | | | | | | | | | | | | | |
| Glycerol | 100 | | | | | | 2.00 | 2.00 | | | | 2.00 | |
| 245 Fluid | 100 | Dow Corning | 61.25 | 70.00 | 43.75 | | | | | 70.00 | 43.75 | | |
| 200 Fluid | 100 | Dow Corning | 29.00 | 19.00 | 30.00 | | | | | 20.00 | 45.00 | | 70.00 |
| (20 Cst) | | | | | | | | | | | | | |
| Water | 100 | | | | | 93.75 | 90.53 | 93.00 | 97.45 | | | 94.75 | |

Particles

| | | | | | | | | | | | | | |
|---|---|---|---|---|---|---|---|---|---|---|---|---|---|
| Micro Titanium | 100 | Tri-K | 1.00 | | | | | | | | 5.00 | | |
| Dioxide | | | | | | | | | | | | | |
| MT-100SA | | | | | | | | | | | | | |
| MSS-500/3N | 100 | Kobo | | 1.00 | 6.65 | | | | | | | | 10.00 |
| Silica | | | | | | | | | | | | | |
| Neosil CT11 | 100 | Crossfield | | | | 1.00 | 1.00 | 1.00 | | | | | |
| Titanium | 100 | Whittaker | | | | | | | 0.30 | | | | |
| dioxide | | | | | | | | | | | | | |
| Ganzpearl | 100 | Presperse | | | 10.00 | | | | | | | | 15.00 |
| GMP-0820 | | | | | | | | | | | | | |
| Mica S-2 | 100 | Kobo | | | 3.35 | | | | | | | | 5.00 |
| Total | | | 100 | 100 | 100 | 100 | 100 | 100 | 100 | 100 | 100 | 100 | 100 |

TABLE 6

| Properties | | | |
|---|---|---|---|
| | | Between: | and: |
| Desired property | Fn | 0 | 0.1 [N] |
| ranges: | n @ 1 1/s | 10 | 100 [Pa · s] |
| | n @ 10,000 1/s | 0.01 | 0.3 [Pa · s] |
| | 60° gloss | 0 | 10 [g · u] |
| | Opacity | 3 | 6 [%] |

| | | Fn | n @ 1 1/s | n @ 10,000 1/s | 60° gloss | Opacity |
|---|---|---|---|---|---|---|
| Composition # | Composition | [g] | [Pa · s] | [Pa · s] | [g · u] | [%] |
| Pola Daily Cosme | Foundation | 62.67 | 198.7 | 0.2553 | 3.5 | 2.88 |
| Shiseido Asplir | Foundation | 28.35 | 175.0 | 0.3995 | 4.4 | 3.14 |
| Shiseido Selfit | Foundation | 4.46 | 22.65 | 0.0518 | 24.6 | 3.76 |
| 1 | 80% DC9040, 1% TiO2 | 3.38 | 15.06 | 0.1219 | 2.0 | 4.70 |
| 2 | 80% Velvesil, 1% TiO2 | 7.63 | 63.99 | 0.1680 | 1.5 | 4.40 |
| 3 | 50% DC9040, 20% soft | 0.50 | 29.44 | 0.1419 | 3.4 | 4.56 |
| | focus particle mix | | | | | |
| 4 | 20% Silsoft, 1% Neosil | 6.33 | 13.73 | 0.0366 | 4.4 | 3.54 |
| 5 | 2.9% Flexan II, 1% Neosil | 4.35 | 17.14 | 0.0127 | 1.6 | 4.62 |
| 6 | 30% Styleze, 1% Neosil | | | | 14.7 | 1.57 |
| 7 | 6.9% Avalure, 0.3% TiO2 | 7.74 | 3.845 | 0.0362 | 29.2 | 2.06 |
| 8 | 80% DC9040 | 2.42 | 2.686 | 0.1222 | | 3.31 |
| 9 | 50% DC9040, 5% TiO2 | −4.53 | 59.03 | 0.0460 | 3.0 | 7.70 |
| 10 | 30% Styleze | 22.22 | 0.6967 | 0.0385 | 85.8 | 0.90 |
| 11 | 30% soft focus particle mix | | | | 4.7 | |

### EXAMPLE 2

The effect of the compositions 1-11, as well as the Pola and Shiseido products, on the appearance of pore size was evaluated in a sensory panel, using the Pore Ruler technique described hereinabove. The Pore Ruler grade change was generally evaluated compared with application of demineralized water. In some cases, it was evaluated compared to the formulation vehicle, that is the formulation without the polymer and particulates, or compared to other formulations.

TABLE 7

| | | Pore ruler | number | | |
|---|---|---|---|---|---|
| Composition | | grade | of | compared | |
| No. | Composition | change | subjects | against | P-value |
| Pola Daily | Foundation | −1.25 | 14 | water | 3.7 × 10⁻⁴ |
| Cosme | | | | | |
| Shiseido Selfit | Foundation | −2.23 | 13 | 3 | 1.6 × 10⁻⁵ |
| 1 | 70% DC9040, 1% TiO2 | −2.78 | 16 | water | 1.6 × 10⁻⁴ |
| 3 | 50% DC9040, 20% soft | −4.23 | 13 | Selfit | 1.6 × 10⁻⁵ |
| | focus particle mix | | | | |
| 4 | 20% Silsoft, 1% Neosil | −1.93 | 15 | water | 7.6 × 10⁻⁴ |
| 5 | 2.9% Flexan II, 1% Neosil | −1.96 | 23 | vehicle | 0.0172 |
| 6 | 30% Styleze, 1% Neosil | −1.00 | 12 | water | 0.0089 |
| 7 | 6.9% Avalure, 0.3% TiO2 | −1.62 | 16 | water | 0.023 |
| 10 | 30% Styleze | −0.10 | 15 | water | 10⁻¹⁰ |
| 11 | 30% soft focus particle mix | −1.97 | 15 | water | 0.0084 |

It can be seen from this Example that, the compositions within the scope of the present invention reduce pore size appearance, to the extent of at least about 2 unit improvement as measured on the Pore Ruler.

The foregoing description and examples illustrate selected embodiments of the present invention. In light thereof variations and modifications will be suggested to one skilled in the art, all of which are within the spirit and purview of this invention. It is intended that all of these modifications and variations be within the scope of the present invention as described and claimed herein, and that the inventions be limited only by the scope of the claims which follow, and that such claims be interpreted as broadly as is reasonable. Throughout this application, various publications have been cited. The entireties of each of these publications are hereby incorporated by reference herein.

1. A cosmetic composition comprising:
(a) about 0.01 to about 30% by weight of the composition of a crosslinked polysiloxane elastomer;
(b) about 0.1% to about 25% of light scattering particles; and
(c) a cosmetically acceptable vehicle;
wherein said composition has:
a viscosity of at room temperature of about 10 Pa.s to about 100 Pa.s at a shear rate of 1 1/s; and about 0.01 Pa.s to about 0.3 Pa.s at 10,000 1/s;
an Opacity of about 3% to about 6%;
a 60 degrees Gloss of up to about 10 g.u.;
a normal force of up to about 0.1 N at a shear rate of 10,000 1/s; and
said composition resulting in an improvement of appearance of facial pores of at least about 2 as measured by the Pore Ruler.

2. The composition of claim 1, wherein the composition further comprises a silicone oil.

3. The composition of claim 1 wherein the light scattering particles are selected from the group consisting of titanium dioxide, titanium coated mica, silica, talc, PMMA, nylon, microcrystalline cellulose, and mixtures thereof.

4. The composition of claim 1, wherein the light scattering particles are selected from the group consisting of titanium coated mica, silica, PMMA, and mixtures thereof.

5. The composition of claim 1, wherein said elastomer is present in an amount of about 1% to about 10% by weight of the composition.

6. The composition of claim 1, wherein said light scattering particles are present in an amount of about 1% to about 10% by weight of the composition.

7. The composition of claim 1, wherein said light scattering particles have a particle size of less than about 30 micrometers.

8. The composition of claim 1, further comprising a thickener in an amount of up to about 2% by weight of the composition.

9. A method of reducing the appearance of skin pores comprising applying to the skin a composition according to claim 1.

10. The method of claim 9, wherein said improvement of appearance of facial pores is about 4 as measured by the Pore Ruler.

11. A cosmetic composition comprising:
(a) about 0.01 to about 10% by weight of the composition of a water based polymer;
(b) about 0.1% to about 25% of light scattering particles; and
(c) a cosmetically acceptable vehicle;
wherein said composition has:
a viscosity of at room temperature of about 10 Pa.s to about 100 Pa.s at a shear rate of 1 1/s; and about 0.01 Pa.s to about 0.3 Pa.s at 10,000 1/s;
an Opacity of about 3% to about 6%;
a 60 degrees Gloss of up to about 10 g.u.;
a normal force of up to about 0.1 N at a shear rate of 10,000 1/s; and
said composition resulting in an improvement of appearance of facial pores of at least about 2 as measured by the Pore Ruler.

12. The composition of claim 11, wherein the light scattering particles are selected from the group consisting of titanium dioxide, titanium coated mica, silica, talc, PMMA cross-polymer, nylon, microcrystalline cellulose, and mixtures thereof.

13. The composition of claim 11, wherein the light scattering particles are selected from the group consisting of titanium coated mica, silica, PMMA, and mixtures thereof.

14. The composition of claim 11, wherein said polymer is present in an amount of about 1% to about 10% by weight of the composition.

15. The composition of claim 11, wherein said light scattering particles are present in an amount of about 0.5% to about 10% by weight of the composition.

16. The composition of claim 11, wherein said light scattering particles have a particle size of less than about 30 micrometers.

17. The composition of claim 11, further comprising a thickener in an amount of up to about 2% by weight of the composition.

18. A method of reducing the appearance of skin pores comprising applying to Is the skin a composition according to claim 11.

19. The method of claim 18, wherein said improvement of appearance of facial pores is about 4 as measured by the Pore Ruler.
